(19) United States
(12) Patent Application Publication
(10) Pub. No.: US 2024/0369241 A1
(43) Pub. Date: Nov. 7, 2024

(54) Ventilation and Air Treating Devices

(71) Applicant: Synergetics Pty. Ltd., West Melbourne, Victoria (AU)

(72) Inventors: David Collins, Parkville (AU); Callum Maltby, Newtown (AU); James Forsyth, Murrumbeena (AU); Malavika Haribabu, Point Cook (AU); Elin Olsson, South Yarra (AU); Nitheesh George, Essendon (AU)

(21) Appl. No.: 18/567,422

(22) PCT Filed: Jun. 10, 2022
(86) PCT No.: PCT/IB2022/055422

§ 371 Date: Dec. 6, 2023

(30) Foreign Application Priority Data
Jun. 10, 2021 (AU) 2021901755

(51) Int. Cl. F24F 3/163; F24F 13/28

(52) U.S. Cl. CPC F24F 3/163; F24F 13/28

(57) ABSTRACT

Apparatus and methods for treating air from a working space 22 that lies above a work surface 20 and in front of a user 8 of the working space. Air is drawn through an air inlet 28 into a fan/filter unit 26, the air inlet in use being located on an opposite side of the work surface from the user and drawing air from the working space above the work surface and at least partly from below the level of the user's face. Air after passing through the fan/filter unit is expelled through an air outlet 30 outside the working space. Additionally a workstation 498 incorporating the apparatus.

### FIELD

The invention provides devices adapted to limit the concentration of droplets, aerosols and particulates in air in a working space above a work surface in front of a user of the work surface, whereby to limit potential for spread of disease from the user to other persons. A particular application is to computer workstations.

### BACKGROUND

Certain diseases of viral or bacterial type can be transmitted from an infected person to an uninfected person through air, when the uninfected person breathes in airborne droplets and/or aerosol particles containing the causal virus or bacterium breathed out or otherwise expelled by the infected person. An important example is the disease caused by the so-called COVID-19 virus that reached pandemic proportions in the year 2020. (COVID-19 disease may also infect persons who touch surfaces in which the virus is present. One cause of such surfaces bearing the virus is settling of droplets and aerosol particles onto them.)

COVID-19 disease affects the respiratory tract of infected persons. Accordingly, droplets and aerosol particles may be simply breathed out by persons having the infection or expelled by coughing or sneezing.

It is very common today for people to spend significant periods using computers or computer terminals and/or watching computer screens. This may be in a space (e.g. room) such as an office or other workplace or in a residence, and these environments may contain multiple people, causing a risk of spreading of diseases that are spread by airborne means due to movement of air within the space. The risk may be exacerbated by air conditioning or other air circulating means operating in the space.

This invention is directed particularly to limiting the potential for spread of diseases borne in the air space between a computer or computer terminal or computer screen (hereinafter “computing equipment”) and a user thereof, by removing at least a portion of the air and droplets or particles therein from the space between the user and the computer or terminal or screen. That is, removal of potentially infective material is carried out as nearly as possible at its source. A user may simply breathe or cough or sneeze and so place such material into the air space.

It is known from experiments that the momentum of a human sneeze or cough largely dissipates within about 0.8 to 0.9 metres from the source (mouth) but droplets and particularly aerosol particles may remain suspended in air long enough to be carried much further by general air movement within the space. The wearing of a mask can limit the spread of air that is breathed or coughed out, but most people prefer not to wear them for significant periods and some people with pre-existing ailments such as asthma or chronic obstructive pulmonary disease (COPD) are unable to.

Importantly, embodiments of the invention can largely avoid interfering with ordinary use of the computing equipment.

While particularly directed to applications involving the use of computing equipment, it is not intended that other potential applications to similar situations be excluded.

### DISCLOSURE OF THE INVENTION

Referring to the attached Figures, FIG. 1 shows schematically a side view of a person 300 (user) using a computer as is typical in offices. A computer monitor assembly 302 is placed on an upper surface 304 of a desk, table or the like 306 at a distance from the user's face 308. At least some of that part of the upper surface 304 of the desk or table between the monitor assembly 302 and the user 300 is to be understood here as a work surface, where the user 300 may place items such as a keyboard assembly 310, as shown. The space 312 above the work surface 304, and in front of the user 300 is to be understood here as a working space for the user 300.

Item 314 in FIG. 1 represents air that enters the working space when the user 300 sneezes or coughs or simply breathes out. Note that references to removal of “air” from the working space here and elsewhere in this specification are to be interpreted as references to atmospheric air that also contains gas exhaled, coughed, sneezed or otherwise expelled by the user, and droplets and aerosol particles entrained in that “air”.

The essence of the invention is to draw air directly from the working space 312 above work surface 304 (both as interpreted in the way set out above) and treat it, for example by filtering, to remove (or reduce the concentration of) exhaled droplets, aerosols or particles that may be able to infect persons with disease or that are otherwise noxious. As air is drawn from the working space 312 air from the general surroundings is drawn into working space 312 so that it becomes a ventilated space. (Both terms, “working space” and “working space” will be used herein.) In some embodiments the treated air is returned into a space (for example a room) containing the working space 312. More preferably, this returning of treated air is to be done in such a way as to minimize rebreathing of the air by the user 300 and also by persons in the user's vicinity.

Note that other devices for treatment of air (for example by adding material to scent the air) can be used where practicable, in addition to a filter.

FIG. 2 shows the same scene as FIG. 1 (the user 308 now represented by his or her head only) and shows an air inlet 316 (represented schematically) located so that the work surface 304 is between firstly the monitor assembly 302 and air inlet 316 and secondly the user 308 (i.e. between chain dotted lines 318 and 320 as drawn), and so that air from working space 312 is drawn into air inlet 316 below the height of the user's face 308, i.e. below the height represented by chain-dotted line 322. Preferably, the air inlet 316 is at or in or closely adjacent to the work surface 304 and below the screen surface 330. Although FIG. 2 shows the air inlet 316 slightly above the level of work surface 304, other possible arrangements are disclosed below. Arrows 317 indicate air being drawn into air inlet 316.

FIG. 19 shows the same scene as FIGS. 1 and 2, save for the absence computer parts 302 and 310, but now with an upright surface 324 facing the user 300 and on an opposite side of the working space 312 from the user 300. The upright surface 324 is shown as being a surface of an office partition 326 but may instead be a surface of a wall or even of a partition secured to the desk or table 306. Three possible locations for an air inlet 328 are shown schematically. Air inlet 328a is similarly located to air inlet 316 shown in FIG. 2, if the screen surface 330 of monitor assembly 302 is interpreted (as it may be) as in effect amounting to an upright surface similar to surface 324. Alternatively, air inlet 328b is an opening into upright surface 324 (still below the level of the user's face 308 as shown by chain-dotted line 332) and preferably close to the level of work surface 304. Also alternatively, air inlet 328c comprises an opening in the work surface 304 itself on the side of the work surface 304 opposite the user's side. Arrows 334 represent air flowing from working space 312 into air inlet 328a. Any one or more of these alternative air inlet positions may be used. A laptop computer 336 is shown on work surface 304.

The presence of an upright surface such as 324 (or 330 in FIG. 2) is preferred because it limits the degree to which air is drawn into the air inlet(s) such as 328 or 316 from the side of the air inlet(s) remote from the user 300.

The invention may be applied to a laptop computer in itself, as shown in FIG. 20. Laptop computer 338 comprises a screen portion (monitor assembly) 340 and a keyboard portion 342, hinged together at 344. The keyboard 346 amounts to a work surface (analogous to work surface 304) and the screen surface 348 amounts to an upright surface analogous to upright surface 324. An air inlet 350 (represented schematically) may be placed at or close to hinge location 344 to draw air in from a working space 352 between surface 348 and the user 354 and from a height below the user's face 356 and above the (keyboard) work surface 346. Arrows 356 indicate air flowing into air inlet 350.

Generally, embodiments of the invention provide in a first aspect, apparatus for treating air from a working space that lies above a work surface and in front of a user of the working space, the apparatus comprising:
an air handling unit;
a fan that draws air through an air inlet into the air handling unit, the air inlet in use being located on an opposite side of the work surface from the user and drawing air from the working space above the work surface and at least partly from below the level of the user's face;
a filter within the air handling unit through which air drawn into the air handling unit passes; and
an air outlet through which air leaving the filter is expelled, the air outlet being positioned outside the working space.

Preferably, the working space is further bounded in part by an upright surface in front of the user and the work surface is between the upright surface and the user, and wherein the air inlet is positioned at the upright surface or closer to the upright surface than the user.

The upright surface may comprise a screen surface of a computer monitor assembly and at least a portion of the air inlet may in use be positioned below the computer monitor assembly. The air outlet may in use be positioned behind the computer monitor assembly.

In some embodiments, the air handling unit is adapted to lie below the computer monitor assembly and to support the computer monitor assembly. The air inlet may be comprised in the air handling unit. A gap between the computer monitor assembly may be closed by a screen extending between the air handling unit and the computer monitor assembly.

In other embodiments, described below, the upright surface comprises the screen surface of a computer monitor assembly and the air inlet wholly or partially surrounds the screen portion of a computer monitor assembly.

In some embodiments having an upright surface as described above, the upright surface comprises a screen surface of a laptop computer and the working surface comprises a keyboard portion of the laptop computer; and the air inlet is in use positioned on the work surface further from the user than the keyboard of the keyboard portion and below the screen surface. Preferably, the air inlet may in use be positioned at a re-entrant corner between the work surface and a portion of the laptop computer comprising the screen of the laptop computer.

It is preferred, to ensure adequate drawing of air from the working space, that the air inlet is elongate in a direction transverse to the user when the user is facing the work surface.

In further embodiments, the invention provides a work station having a working space that lies above a work surface and in front of a user of the work station, the work station comprising:
an air handling unit;
a fan that draws air through an air inlet into the internal space of the air handling unit, the air inlet in use being located on an opposite side of the work surface from the user and drawing air from the working space above the work surface and at least partly from below the level of the user's face;
a filter within the air handling unit through which air drawn into the air handling unit passes; and
an air outlet through which air outlet air leaving the filter is expelled, the air outlet positioned outside the working space.

Preferably, the working space is further bounded in part by an upright surface in front of the user and the work surface is between the upright surface and the user, and the air inlet is positioned at the upright surface or closer to the upright surface than the user.

The upright surface of the work station, when present, may comprise a surface of a wall or partition.

In work stations embodying the invention, it is preferred, to ensure adequate drawing of air from the working space, that the air inlet is elongate in a direction transverse to the user when the user is facing the work surface. Alternatively, the air inlet may comprise one of at least two air inlets one positioned to the left and one positioned to the right of the user whereby to draw air from both sides of the working space.

In a work station according to the invention, the air handling unit may be positioned at a height less than the height of the work surface. In this case, the air outlet may be directed horizontally and away from a plane that includes the upright surface.

In a further aspect, the invention provides a method for treatment of air from a working space that lies above a work surface and in front of a user of the working space, the method comprising:
by a fan, drawing air through an air inlet into an air handling unit, the air inlet being located on an opposite side of the work surface from the user and drawing air from the working space above the work surface and at least partly from below the level of the user's face;
passing air drawn into the air handling unit through a filter within the air handling unit; and
thereafter expelling air leaving the filter from an air outlet outside the working space.

Preferably in the method, the working space is further bounded in part by an upright surface in front of the user and the work surface is between the user and the upright surface, and including the step of locating the air inlet closer to the upright surface than a face of the user.

Embodiments of the invention as mentioned above are described in more detail below, together with additional embodiments and additional inventions.

In this specification, the word “comprise” and derivatives thereof, including “comprised, “comprises” and “comprising”, when used in relation to elements or steps are intended to mean that the elements or steps are present, but not to preclude the possible presence of other elements or steps. Nothing in this specification is to be taken as an admission as to the state of the art in any jurisdiction.

### BRIEF DESCRIPTION OF THE DRAWINGS

FIG. 1 is a schematic side view of a person using a computer at a work station;

FIG. 2 is the same view as FIG. 1, showing schematically a working space and an air inlet location used in embodiments of the invention;

FIG. 3 is a side view of a computer workstation incorporating a ventilating system according to an embodiment of the invention;

FIG. 4 is a perspective view of the computer workstation as shown in FIG. 3;

FIG. 5 is a perspective exploded view of a ventilating system according to a further embodiment of the invention;

FIG. 6 is a cross sectional view of the ventilating system as shown in FIG. 5, the section taken at station ‘AA’;

FIG. 7 is a cross-sectional view of the ventilating system as shown in FIG. 5, the section taken at station ‘BB’;

FIG. 8 is a side view, partially sectioned, of a computer workstation incorporating a ventilating system according to a further embodiment of the invention;

FIG. 9 is a perspective view of the computer workstation as shown in FIG. 8;

FIG. 10 is a perspective view of a computer monitor fitted with optional side and top flaps;

FIG. 11 is a perspective view of a ventilating system according to a further embodiment of the invention;

FIG. 12 is a perspective view of a laptop computer fitted with the ventilating system as shown in FIG. 11;

FIG. 13 is a schematic view of an arrangement for increasing air flow through a ventilating system according to the invention;

FIG. 14 is a schematic view of a further arrangement for increasing air flow through a ventilating system according to the invention;

FIG. 15 is a schematic view of a of another arrangement for moving air from a working space at a work station;

FIG. 16 is a perspective view of a keyboard assembly that can be used with or in application of the invention;

FIG. 17 is a schematic view of a still further arrangement for increasing air flow through a ventilating system according to the invention;

FIG. 18 is a schematic view of a yet further arrangement for increasing air flow through a ventilating system according to the invention;

FIG. 19 is a schematic side view of a person using a computer at a work station;

FIG. 20 is a further schematic side view of a person using a computer at a work station;

FIG. 21 is a partial side view of a work station and air inlet arrangement according to the invention;

FIG. 22 is a partial side view of a further work station and air inlet arrangement according to the invention;

FIG. 23 is a partial side view of a still further work station and air inlet arrangement according to the invention;

FIG. 24 is a perspective view of a work station that is a further embodiment of the invention;

FIG. 25 is a side view of the work station shown in FIG. 24 showing an air circulation pattern; and

FIG. 26 is a side view of a workstation that is an embodiment of the invention, showing an alternative air inlet arrangement.

### DETAILED DESCRIPTION OF PREFERRED EMBODIMENTS

FIG. 3 shows a first exemplary embodiment of the invention comprising a monitor 14 having a monitor body portion 17 in which is supported a screen 16 and a keyboard 18 on a work surface or tabletop 20. Item number 22 represents the working space (corresponding to space 312 in FIG. 2). Monitor 14 is supported by a support member 24 extending upwards from a fan/filter unit 26 (also to be known as an air handling unit) placed on work surface 20. Fan/filter unit 26 comprises internally a filter (not shown, preferably a HEPA filter) and an electrically driven fan (not shown) that draws air from an air inlet 28 facing the user 8 through the filter and expels the filtered air from an air outlet 30.

To prevent air from escaping from space 22 through gap 32 that extends between monitor body portion 17 of monitor 14 and fan/filter unit 26, an air blocking member in the form of a screen or partition 34 is provided extending between the monitor body portion 17 and the fan/filter unit 26. Partition 34 extends across the width of fan/filter unit 26 (or optionally may be of greater width). Partition 34 is shown as being formed from a suitable plastics material in a concertina form, so as to be adjustable by user 8 to fully close gap 32 and still to permit adjustment of the orientation of monitor 14. However, other ways of closing gap 32 will readily suggest themselves to persons skilled in the art. Any suitable method may be used to secure partition 34 in place—for example only, double sided tape may be used on the upper and lower edges, or the partition 34 may be a permanent part of fan/filter unit 26 and attached by adhesive or adhesive tape to monitor 14 along the partition's upper edge. Partitions are also mentioned below in respect of other embodiments—see for example item 78, 228 and the remarks in this paragraph also apply to them.

Fan/filter unit 26 operates continuously while user 8 is using the monitor and/or keyboard, and is preferably electrically powered. Optionally, fan/filter unit 26 may optionally contain internally one or more ultraviolet light sources to treat air flowing therethrough to enhance disinfection of the air.

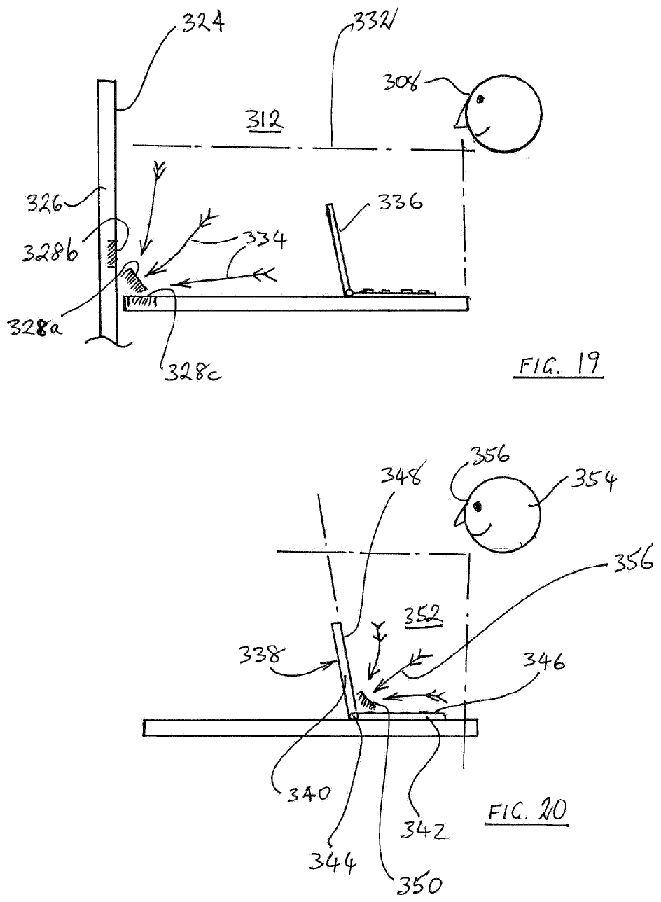

Inlet 28 is located at the front of fan/filter unit 26 and below monitor 14 (i.e. in a location described as desirable in the text relating to FIGS. 2 and 19) because it has been found that this location is particularly well suited to efficient drawing of air from space 22 and so that fan/filter unit 26 can be located conveniently underneath monitor 14, occupying little of the area of work surface 20.

Partition 34 and the front surface of monitor 14 in combination form an upright surface 37 with air inlet 28 at the bottom of that upright surface, the significance of the upright surface 39 being as described above in relation to upright surface 324 shown in FIG. 19. In effect the work surface 20 and upright surface for a re-entrant corner with the air inlet 28 at that re-entrant corner. Partition 34 limits leakage of air through gap 32 instead of air inlet 28.

Outlet 30 is shown as expelling filtered air upward behind monitor 14 as shown by arrow 36. This is optional, but has been found, surprisingly, to have an advantage in some applications, in that continuous operation can cause, in otherwise still air conditions, a circulatory flow pattern (shown by arrows 38) to develop in which treated air directed upward tends to rise behind monitor 14 and then downward into the space 22 in front of screen 16 and toward inlet 28. This flow pattern enhances the ventilation of working space 22. Partition 34 enhances separation of working space 22 and the region behind monitor 14.

FIGS. 5 to 7 show another embodiment of the invention.

In this example, an inlet device 44 is secured to the front of a monitor body portion 39 of a computer monitor 40 having a screen 42. Inlet device 44 is in the form of a frame having elongate air inlet portions that extend around the periphery of screen 42, without significantly encroaching on any image displayed on screen 42. Each elongate air inlet portion of device 44 is hollow and has an opening in the form of a slit 45 through which air may enter from a working space 46 in front of screen 42 into an internal space 48 of device 44. Connected to device 44 is a conduit 50 (shown as a flexible tube, but other suitable forms of conduit are possible) through which air from the internal space 48 is continuously drawn into a fan/filter unit (air handling unit) 52. Although not shown, conduit 50 may in alternative embodiments be one of several conduits positioned so as to limit variation of flow into device 44 around the screen periphery. FIGS. 6 and 7 are cross-sectional views at typical stations on frame-forming portions of device 44. Device 44 includes one elongate air inlet portion 44a that has an internal plenum chamber 54 in fluid communication with internal space 48. Although not shown, Portion 44a of device 44 may also have one or more openings arranged along its length to draw air directly from space 46 into plenum chamber 54, without passing through slit 45, to enhance collection of air near the bottom of monitor 40. Although device 44 is shown as having four elongate air inlet portions, it may optionally have a lesser number (for example the uppermost one could be omitted). Air inlet portion 44a, however, is the key one as it draws in air at a position just below the screen 42 (and below the level of the user's face (not shown)).

Fan/filter unit 52 contains a filter (not shown, preferably a HEPA filter) suitable for removing droplets, aerosol particles and other particulate matter from air drawn into the fan/filter unit 52, and a fan (not shown) powered by an electric motor (not shown). Air is drawn from the working space 46 through device 44 and through conduit 50 and fan/filter unit 52. Fan/filter unit 52 also has an air outlet 56 for air that has passed through its fan and filter. This may optionally direct treated air upwards and be located behind monitor 40.

Inlet device 44 can be secured to monitor 40 in any suitable manner, permanently or temporarily, but is shown in FIGS. 5 and 6 as held in place by clips 58. The monitor 40 in FIG. 5 is supported on a stand 60. As with the embodiment shown in FIGS. 3 and 4, it is desirable that any gap beneath the monitor 40 and the surface (not shown) on which it stands is blocked to limit or prevent escape of air from the working space 46. This can be done in the same way as in the arrangement of FIGS. 3 and 4, by a partition (not shown) but for example similar to partition 34, extending downward from element 44a of device 44.

Although fan/filter unit 52 is not shown in any particular location relative to monitor 40, it may be in a similar form to fan/filter unit 26 shown in FIGS. 3 and 4 and replace stand 60, supporting and sitting below monitor 40. In still another alternative arrangement, fan/filter unit 52 may be similar to fan/filter unit 26 except for not having upright 24, with stand 60 simply placed on top of it.

Still another embodiment of the invention will now be described, by reference to FIGS. 8 and 9. In this embodiment, a monitor body portion 61 of a monitor 62 is housed and secured in an open-fronted boxlike enclosure 64 that in turn is supported above a work surface such as a work surface or tabletop 66 by a fan/filter unit (air handling unit) 68. The enclosure 64 is sized and shaped so that there is a gap 70 between at least a portion of the monitor body portion 61 and outer edge 72 of the enclosure 64. In FIG. 9, the gap 70 is shown as extending completely around the monitor edge 72 including below the screen 76. Air is drawn from the interior of enclosure 64 and therefore through the gap 70 so as to be removed from the working space 74 in front of the screen 76 of the monitor 62. This drawing of air from enclosure 64 is by means of fan/filter unit 68 as for previously defined embodiments, fan/filter unit 68 comprising a filter (again, preferably a HEPA filter, not shown) and a fan (not shown). The fan/filter unit 68 is placed on the tabletop or other work surface 66. An upright member 94, shown partly in section, connecting the fan/filter unit 68 to the enclosure 64 is hollow, and provides a conduit for movement of air from the interior of enclosure 64 into the fan/filter unit 68. Gap 96 between enclosure 64 and the fan/filter unit 68 is preferably closed by a partition 78, as for the embodiments shown in FIGS. 3, 4 and 5, and for the same reasons.

Filtered air is expelled from fan/filter unit 68 through an outlet 80. Although not essential, outlet 80 is advantageously arranged to direct outlet air upward behind enclosure 64, to encourage the kind of circulatory air flow pattern mentioned in relation to the embodiment of FIGS. 3 and 4. Arrows of the types numbered 82 show generally the airflow out of the working space 74 between user 8 and screen 76.

Although not shown, it is possible for fan/filter unit 68 to be provided with one or more additional air inlets on its front panel 84 to enhance the drawing of air from working space 74. Air drawn through such inlets would flow directly to the filter in fan/filter unit 68.

The incorporation of hollow upright member 94 is optional. There may be provided instead a simple stand for monitor 62 on top of fan/filter unit 68 with a separate conduit (not shown for air to be directed from within enclosure 64 to fan/filter unit 68.

FIG. 10 shows a monitor 90 fitted with side-mounted flaps 92 and a top-mounted flap 98. Any or all of flaps 92 and 98 may be fitted, as an option, to the monitors 14 or 40 so as to enhance control of the movement of air into or out of the working spaces 22 or 46. For example side-mounted flaps 92 may partially limit lateral movement of air. Flaps 92 and 98 may be mounted to monitor 90 by any suitable means such as clips or adhesive for example, and preferably would be hinged for manual adjustment by a user to a desired position. Flaps 92 and 98 may also assist in control glare on the screen 100 and enhance privacy for users. For the embodiment shown in FIGS. 8 and 9, flaps (not shown) like flaps 92 and 98 could be added to enclosure 64. Any of flaps 92 and 98 may each if desired be made of several pieces, slideable or telescoping relative to each other to allow adjustment by a user of their size.

In all of the described embodiments, it is not essential to have the fan/filter unit (such as 14, 26 or 68) actually on the working surface as described. It will readily be appreciated that they could instead be located on a floor surface (for example) and connected to separate air inlet devices by suitable conduits, so as to draw air from the working spaces in front of the screens (such as 16, 42, 76). This may assist in limiting noise levels.

An embodiment of the invention applicable to some laptop computers will now be described, by reference to FIGS. 11 and 12. The embodiment is consistent with the description above in relation to FIG. 20. A ventilating device 110 is shown and may be used with a laptop computer 110 having a land (surface) 118 between firstly the upper portion 113 comprising screen 114, and secondly the keys 115 on lower portion 116. Ventilating device 110 comprises a duct 120 having air inlet openings 122, duct 120 being in fluid communication with a filter/fan unit 124 through a connection 121, which may be made flexible to accommodate variable laptop geometries. Filter/fan unit 124 contains (not shown) a filter (again, preferably a HEPA filter) and a fan for drawing air in through openings 122, through the filter and expelling the filtered air out through an outlet 126. When laptop computer 112 is placed on a surface (not shown) duct 120 is placed on land 118 as shown in FIG. 12. Duct 120 may be secured in place by any suitable means such as clips 128, or other demountable fasteners (not shown) such as hook-and-loop fastener pads, suction cups, or weak adhesive, or simply weighted down.

Ventilating device 110, in use, draws air into openings 122 from a working space between a user and the screen 114, as in the previously described embodiments. It will be noted that the relative locations of screen 114 and air inlet openings 122 are similar to the relative locations of inlet 28 and monitor 14 as shown in FIGS. 3 and 4, whose surprising effectiveness was mentioned previously.

A device such as device 110 is preferably small enough to be conveniently taken about with a laptop computer as an accessory.

It will also be appreciated that a laptop-style computer (not shown) could be made with at least an air inlet actually incorporated in the computer, that air inlet being located similarly relative to the computer's screen and keyboard to the location of the air inlet 122 of device 110, i.e. close to the point where the keyboard and screen portions are hinged together. A suitable fan or fan and filter could be incorporated in the laptop computer or alternatively the laptop computer having such an integral air inlet could be provided with an air outlet for connection of a separate fan/filter unit. Such fan/filter unit could itself be similar to the fan/filter unit 26, in being adapted for the laptop computer to sit on top of the laptop computer itself.

FIG. 16 and its supporting text below disclose an arrangement in which an air inlet comprises openings close to the banks of keys of a keyboard or even the openings between the keys themselves. The laptop computer embodiments disclosed in the previous paragraph could be provided with such an air inlet arrangement.

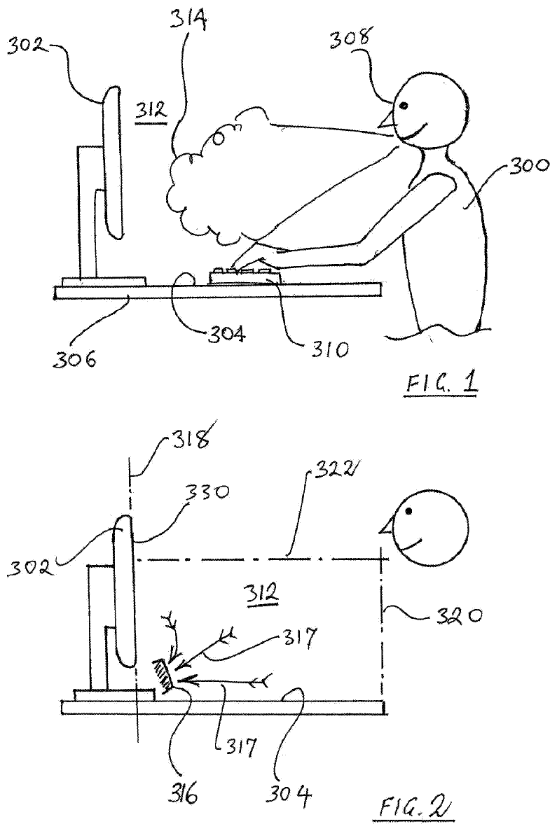

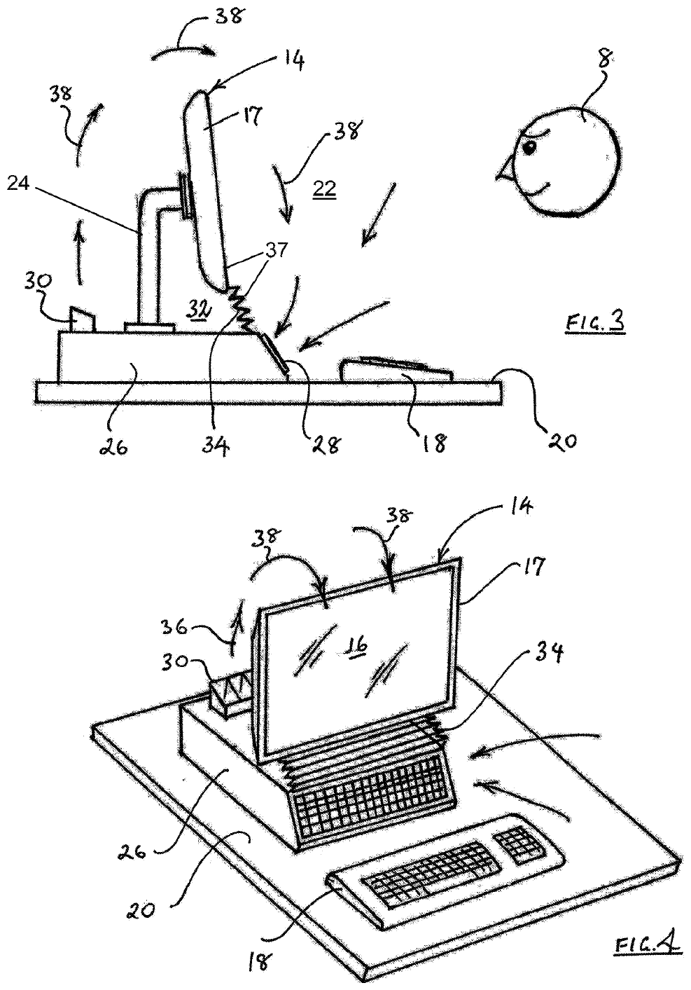

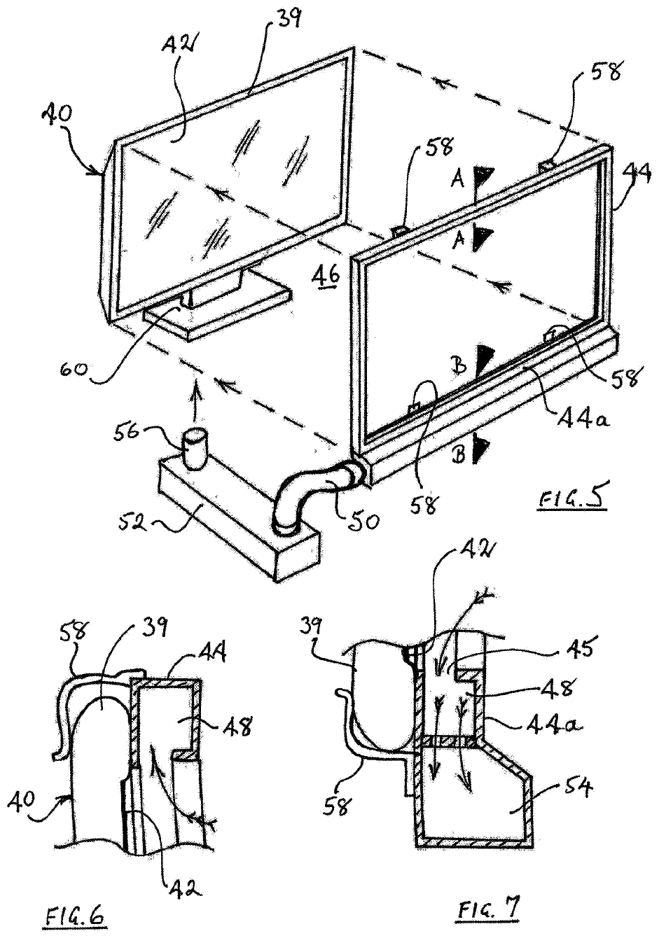

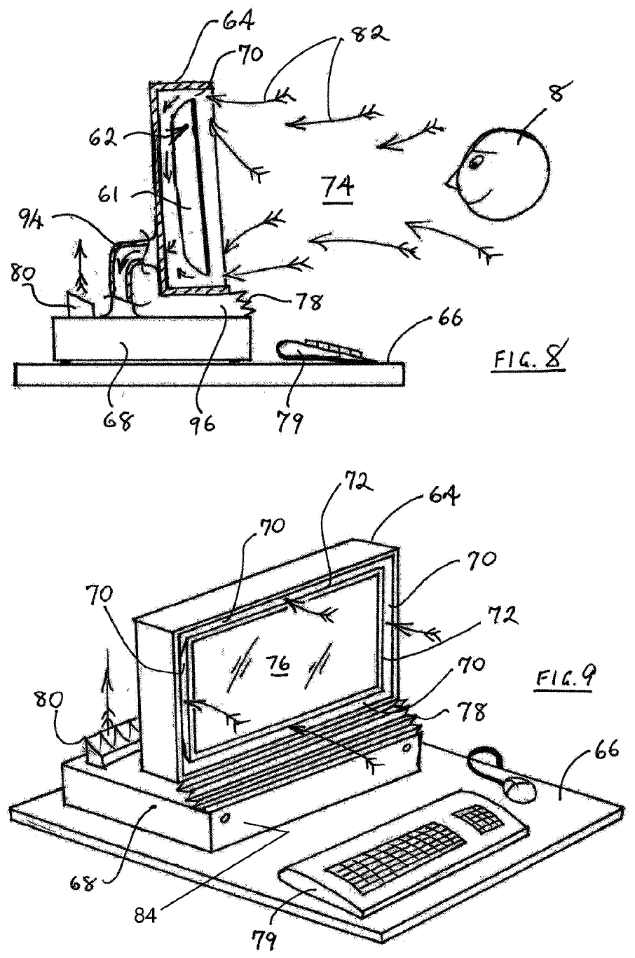

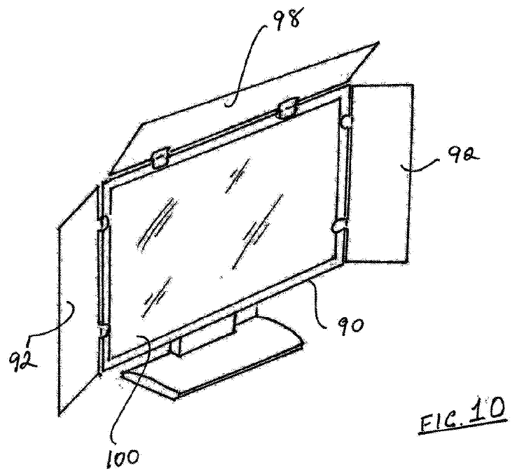

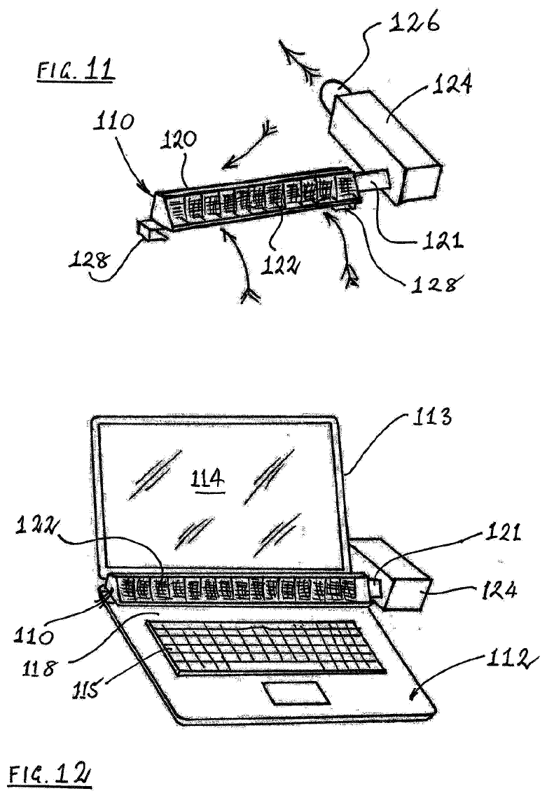

It will be appreciated that all of the devices described by reference to FIGS. 1 to 12, and 14 to 16, have been shown with air inlets that are elongate in a direction (“transverse direction”) that is transverse to users, when the users are facing the work surface or the computer screen or keyboard (i.e. into and out of the page in the views of FIGS. 1 and 2). This is preferred, to enhance the drawing in of a high proportion of air exhaled or expelled by users, and in general the greater the width of the air inlet(s) the better.

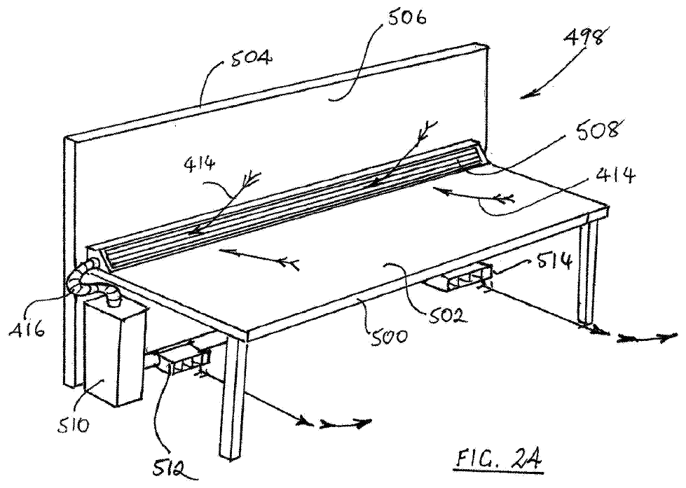

There will now be described a further work station arrangement that more easily lends itself to ventilation of a wider workspace than the arrangements described above. FIG. 24 shows a work station 498 having a desktop 500 with work surface 502 adjoining a partition 504 (that may be part of a wall, or an ordinary office partition, or a partition that is specifically secured to the desktop 500 to provide an “upright surface” 506 of the type mentioned for example in relation to FIG. 19 (see item 324). In work station 498, the upright surface 506 is not a surface that comprises a portion of a computer screen such as 16 or 76, or a gap-closing partition such as 34, 78. A computer monitor can be placed on work surface 502 in front of upright surface 506. An air inlet 508 is located in the re-entrant corner between work surface 502 and upright surface 506 and is elongate in the transverse direction (as defined elsewhere herein), extending along an edge of the work surface 502 remote from where a user (not shown in FIG. 24) would be.

FIG. 23 shows how the air inlet 508 can be provided. FIG. 23 shows desktop 500, user 408 and partition 504. A plenum chamber 501 with air inlet 508 comprising openings into plenum 501 is placed or secured in the upper re-entrant corner formed where the partition 504 and desktop 500 meet, and air drawn into the plenum chamber 501, as indicated by arrows 414 travels to the air handling (fan/filter) unit 510 through a duct 416. Plenum chamber 501 is of comparable width to desktop 500.

Air handling unit 510 is floor mounted and has air outlets 512 and 514 that direct treated air approximately horizontally away from partition 504. This can have an advantage illustrated in FIG. 25. Subject to the state of the air in the vicinity of work station 498, it is possible that a recirculation of air as shown by arrows 516 can build up, returning treated air to the working space above desktop 500, and protecting to some extent persons in the vicinity. A computer monitor 518 is shown in use, and does not need to have the gap 520 between its lower edge desktop 500 sealed. Better flow of air to air inlet 508 occurs when the gap 520 is left open.

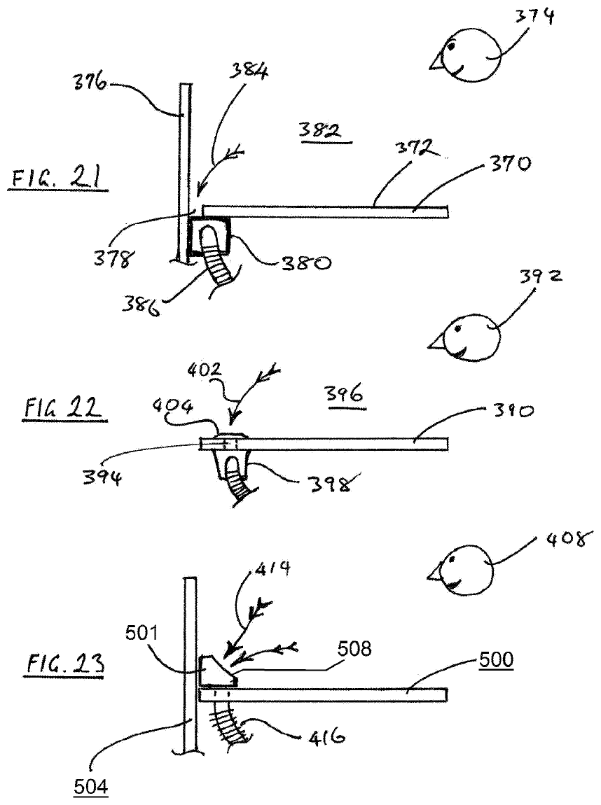

FIGS. 21 and 22 show other ways in which an air inlet arrangement similar to air inlet 508 can be provided in a work station similar to work station 498. FIG. 21 shows a desktop, table top or the like 370 with a work surface 372 and a user 374. A partition 376 or wall is shown, and there is a gap 378 between partition 376 and desktop 370. Mounted to desktop 370 (and/or partition 376) below gap 378 is a plenum chamber 380, elongate in the transverse direction and having a slit (not shown) in registration with gap 378 so that air can be drawn by an air handling unit (not shown) from plenum chamber 380 and hance through gap 378 and the slit from working space 382, as indicated by arrow 384. The gap 378 and plenum chamber 380 can be of a length the same or similar as the width (in the transverse direction) as desktop 370. Air leaving plenum chamber 380 passes to the air handling unit (not shown) by a duct 386.

FIG. 22 shows a desktop 390 with no partition, and a user 392. Several transversely elongate slits 394 are provided in the desktop 390 and air from working space 396 is drawn downward through them as indicated by arrow 402 into a plenum chamber 398, elongate in the transverse direction and via duct 400 to the air handling unit (not shown). A perforated cover 404 may be placed over the slits 394. Plenum chamber 398 may be of comparable width to desktop 390.

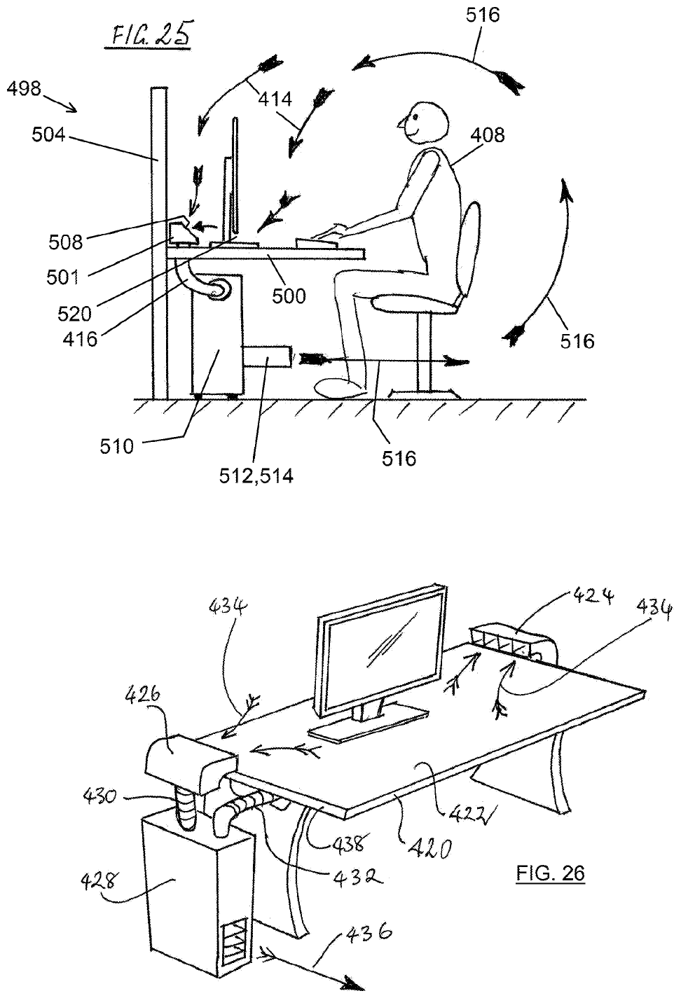

FIG. 26 shows still another alternative arrangement that enables air to be drawn from a transversely wide working space. Instead of an air inlet extending transversely across the working space, at least two inlets 424 and 426 may be provided, one on each of the opposite sides of a desktop 420 having work surface 422. Air is drawn into air inlets 424 and 426 (as indicated by arrows 434) and by ducts 430 and 432 into air handling unit 428 shown as being placed on the floor, i.e. at a lower level than work surface 422. Treated air is shown (by arrow 436) flowing away from air handling unit 428 in an approximately horizontal direction, parallel to a side 438 of the desktop.

Fan/filter units 26, 52, 68 and 124 are all intended to be able to operate continuously when their respective associated monitors are in use. However, the invention may further comprise means for sensing the presence of a user (eg user 8) and automatically switching the filter/fan unit on or off accordingly. This may be done with any suitable sensors, such as infra-red sensors to detect body heat, or through supply of power from the same source as the monitor or controlled by the computer equipment driving the monitor through detection of switching on or off, or execution of keystrokes, or the like as desired. Of course, the fan/filter units may be simply turned on and off by a user operating an electrical switch (not shown) to start the fan (not shown).

An enhancement for ventilating devices as disclosed above will now be described. Particularly when a user such as user 8 has a respiratory ailment, it is possible he or she may from time-to-time sneeze or cough and as a desirable option, means may be provided to temporarily increase the flow rate of air into the device in response to such event. This will ideally be actuated automatically. Possible means for providing for any of the described embodiments with such increased flow or “surge” capacity and automatic actuation of that surge capacity are disclosed below.

A cough or sneeze can be detected with a reasonable degree of reliability using a sound sensor (microphone and compatible electronic control system, not shown) located in or near the working space, and actuation of the devices described below for then providing surge capacity can be readily provided by monitoring the sensor's (by the computer being used if desired) output for a sudden increase of sound level. Alternatively or additionally, provision may be provided for a user to demand “surge” capacity by operation of an electrical switch, (not shown) to actuate electrical equipment as described below. Still another possibility is to incorporate software into the computer being used by the user so that surge capacity can be actuated through the keyboard or mouse of that computer.

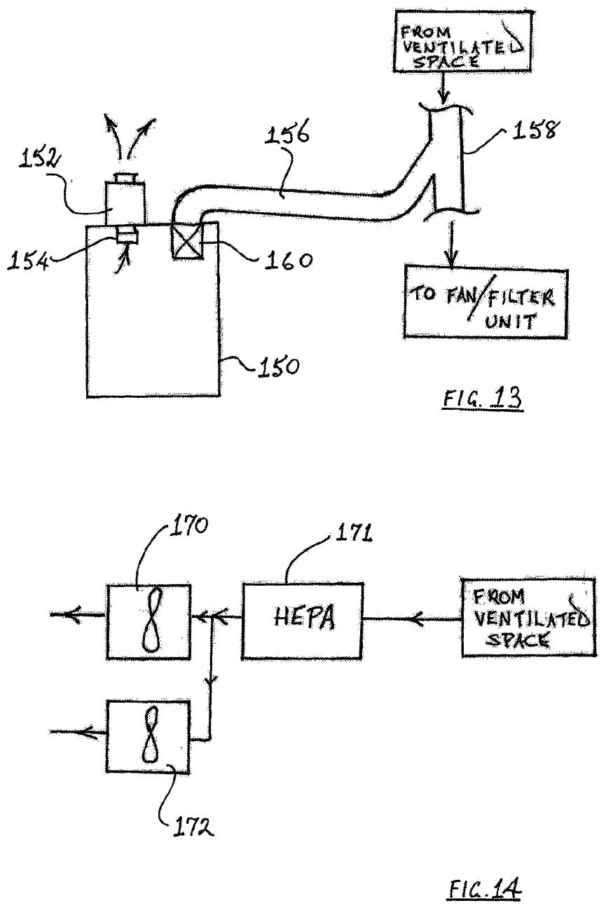

FIG. 13 shows one arrangement for providing “surge capacity” so that in the event of a user sneezing or coughing suddenly, air is removed at a temporarily increased rate from the working space. A vessel 150 is provided with a motor-driven air pump 152 that operates automatically to reduce internal pressure in the vessel 150 until a specified degree of vacuum is reached and thereafter operates when and if the pressure in the vessel rises beyond a specified threshold. A check (that is, one-way) valve 154 is provided to limit or prevent air movement into vessel 150 when the air pump is not operating. Vessel 150 is also connected by a gas conduit 156 to a suitable point in a flow path 158 through which air is sucked from the working space (such as 22, 46 or 74) and has a valve 160 adapted to be opened quickly when a user's cough or sneeze is detected, or when actuated by the user, and electrically operated to open. This sudden connection of the partially evacuated vessel 150 to flow path 158 causes a temporary increase in the flow of air from the working space through conduit 156. Increased pressure in vessel 150 is then sensed and triggers the valve 160 to close and air pump 152 to operate, thus partially evacuating vessel 150 again.

Instead of electrically operated air pump 152, a simple foot- or hand operated pump (not shown) could instead be operated by a user to evacuate container 150 ready for later actuation of valve 160 as described above. (This requires appropriate sealing of container 150 so that air does not leak into container 150 after it has been evacuated to such an extent that surge capacity is significantly reduced.)

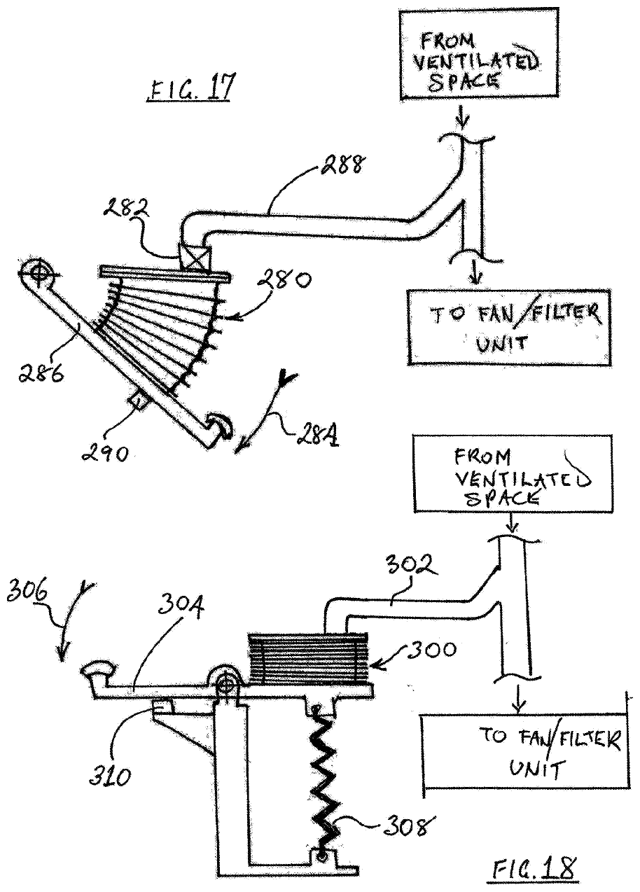

Instead of evacuating a container (such as container 150) of fixed volume, it is possible instead to use a container of variable volume to achieve an evacuated space into which air can be drawn quickly. FIGS. 17 and 18 show two ways in which this can be done. FIG. 17 shows a container 280 that is formed from a flexible and substantially air-impervious material and that, when a valve 282 is in a closed position, can have its internal volume increased by movement (in the direction of arrow 284, by a user's foot or hand or electrically) of a pivoted lever 286, thus creating a degree of vacuum in the container 280. Valve 282 is opened quickly (like valve 160 of FIG. 13) when a user's cough or sneeze is detected, or when actuated by the user, so that air flows into container 280 through duct 288. Afterwards, a catch 290 which holds container in the expanded position shown, is released, lever 284 is moved upward (by the hand or foot of the user or by resilient means such as a spring (not shown)) and air leaves container 280 through valve 282 (or a separate one-way valve (not shown), and the cycle can be repeated.

Container 280 works like a bellows, and is shown as a bendable and extendable tube with rings along its length to prevent it collapsing inward when lever 284 is pushed down. However, other geometries and constructions will readily suggest themselves to persons skilled in the art. Other mechanisms, included electrically operated mechanisms, will also suggest themselves and may be used.

FIG. 18 shows an arrangement in which a variable volume container 300 is expanded in volume suddenly on detection of a cough, sneeze or the like or on actuation by a user, so that air is drawn into container 300 through air duct 302 (equivalent to duct 288 of FIG. 17). Container 300 is shown as of similar construction as container 280, but other constructions may of course be used. Moving a pivoted lever 304 in the direction of arrow 306 causes the volume of container 300 to be minimized and extends a tension spring 308. A releasable catch 310 limits travel of lever 304 and and holds it in the position shown until a user's cough or sneeze or the like is detected or increased airflow is demanded by the user. At that point, catch 310 releases lever 304, and spring 308 quickly contracts so as to increase the volume of container 300 and such air into it through duct 302. Other functionally equivalent mechanisms will readily suggest themselves to persons skilled in the art and can be used. Afterwards, the return of lever 304 to the position shown in FIG. 18 can be by hand- or foot operation by the user or by electrical means.

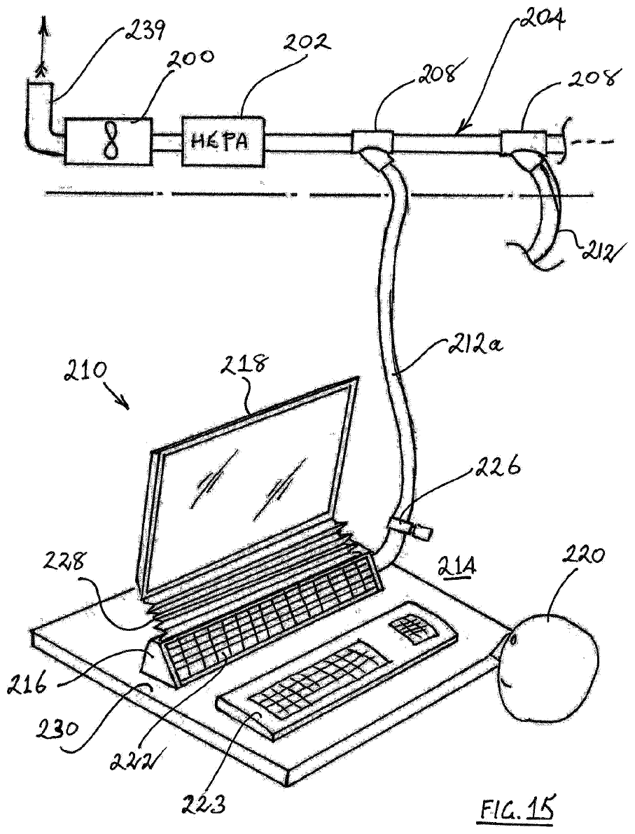

FIG. 14 shows schematically a further alternative arrangement for providing “surge capacity”. In addition to the fan 170 that operates normally to remove air from the working space (fan 170 being for example the fan of one of fan/filter units 26, 52, 68 or even 124 and filter 171 being its associated filter), a second fan 172 is provided that does not normally operate, but that on detection of a cough or sneeze can be operated for a short period with higher capacity than that provided by fan 170 alone. Operation of fans 170 and 172 will require more power to operate than fan 170 alone, and will generally be much noisier, but fan 172 needs only to be operated for a short time to deal with the air expelled in the user's sneeze or cough. It may for example be switched off after a predetermined period has elapsed since detection of the cough or sneeze condition.

In an alternative to provision of a second fan, not shown, only one fan is provided, which on detection of a cough or sneeze condition or when demanded by a user, is temporarily speeded up enough to increase the air flow from the working space. After a period of operation at the higher speed, the fan would revert to operation at a lower speed suitable for ordinary operation.

In essence, the arrangements described above for temporarily increasing air movement from a working space fall into the following classes:
(a) increasing fan capacity temporarily;
(b) connecting a previously evacuated container suddenly to an air conduit removing air from the working space;
(c) suddenly expanding the volume of a container connected to an air conduit removing air from the working space.

The “surge” arrangements described above based on providing temporarily increased fan capacity may be incorporated in the fan/filter units of the embodiments described above or in separate units, as required. Mains power operation of the fan(s) is preferred, although battery operation is also possible if required, and may be appropriate for at least the device shown in FIGS. 11 and 12.

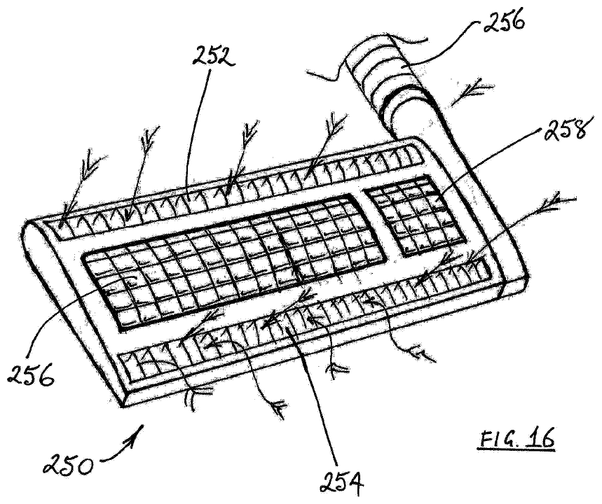

Each of the embodiments shown in FIGS. 3 to 12 comprise fan/filter units that are adjacent to, and associated with, a particular monitor. However, it is possible instead to provide a centralized fan/filter unit to draw air from inlets at multiple locations to provide working spaces as defined herein) at those locations. FIG. 15 is schematic, showing such an arrangement. A fan 200 and filter 202 (preferably a HEPA filter) are provided to draw air from a manifold 204 that extends and is concealed in a space above a ceiling 206, the manifold 204 having connection points 208 for a number of air inlet conduits 212 each of which extends downward from the ceiling 206 to a work station 210. Air is expelled from fan 200 through an outlet 239. (Only two connection points 208 and two inlet conduits 212 are shown, but of course any number can be provided for. Work station 210 is shown in perspective view and is generally similar to that shown in FIGS. 3 and 4, except that instead of fan/filter unit 26 there is provided an air inlet assembly 216 positioned to draw air from working space 214 between monitor 218 and the head of a user 220. Air inlet assembly 216 has an inlet opening 222 positioned similarly relative to monitor 218 as inlet 28 of fan/filter unit 26 is positioned relative to monitor 14. Air inlet assembly 216 has an air duct 224 that directs air drawn into opening 222 into air inlet conduit 212a (which is one of conduits 212). A valve 226 is provided to close off conduit 212a when work station 210 is not in use.

Corresponding to (and optionally similar to) partition 34 of FIGS. 3 and 4, a partition 228 is provided to close a gap between monitor 218 and work surface 230. Partition 228 does for workstation 210 what partition 34 does for the work station shown in FIGS. 3 and 4.

Although the use of a manifold such as manifold 204 has been disclosed by reference to a work station similar to that shown in FIGS. 2 and 3, it is to be understood that the other arrangements described above (eg by reference to FIGS. 5 to 7, 8 and 9 and 11 and 12) could be adapted readily to centralized ventilation in the same way.

Centralized ventilation may have an advantage of reduced noise, because one fan (such as fan 200) can be located remotely from workstations or otherwise have its noise limited more easily than potentially many fans.

The work stations shown in FIGS. 3 to 9 and 15 are shown with conventional keyboard assemblies (eg 18 in FIGS. 3 and 4, 79 in FIGS. 8 and 9 and 223 in FIG. 15) on their respective working surfaces. A further option for enhancing removal of air from the working spaces above these assemblies is shown in a keyboard assembly 250 in FIG. 16. Keyboard assembly 250 has air inlets 252 and 254 that extend across a part of its width, through which air may be drawn from above the assembly 250. Air inlets 252 and 254 are in fluid communication with an outlet duct 256 that in use would be connected to a co-operating inlet (not shown) of fan/filter unit as described above by reference to items 26, 52 68, or to a centralized source of air withdrawal such as the arrangement in FIG. 15 including manifold 204. Relative to a user (not shown in FIG. 16) of the keyboard assembly 250, air inlet 254 is closer to the user, and air inlet 252 is further from the user, than key arrays 256 and 258. It is known that computer keyboards can become fouled by infectious material by touching of the keys and by setting of droplets and particles breathed out or ejected by their users, and the arrangement of assembly 250 addresses the second of these.

Optionally, one of air inlets 252 and 254 could be omitted. Still another possibility (not illustrated) is to provide a keyboard assembly in which air is sucked into one or more spaces within the body of the keyboard assembly through gaps between adjacent keys, the spaces being in fluid communication with ab outlet duct corresponding in function to duct 256.

It is envisaged and preferred that a keyboard assembly such as assembly 250 would not be relied on as the sole source of air movement out of the working space in which it is placed, but rather as an adjunct or enhancement of the arrangements described earlier.

Note that air inlet 252 is similar to air inlet 122 of device 110 of FIGS. 11 and 12. A laptop computer (not shown) may instead be provided with “built-in” air removal arrangements equivalent to that shown for conventional keyboard assembly 250, with air inlets (not shown) in one or both of the positions relative to its key array(s) corresponding to the positions of air inlets 252 and 254 or alternatively with suction through gaps between keys of its key arrays. Such a laptop could be provided with an air outlet for connection to a fan/filter unit as described above to enable the laptop computer itself to be kept reasonably compact.

Where it is desired to provide “surge” capacity as described above, a simple way to do this with centralized ventilation is to specify fan 200 to provide an air flow rate capacity greater than is adequate for normal operation (i.e. in the absence of sneeze or cough conditions) at every work station such as station 210, and provide for valve 226 to be opened wider than usual at a particular work station such as 210 when a sneeze, cough or the like is detected or in response to a user demand. Valve 226 can in such a case be triggered to allow more flow on detection of a sneeze, cough or the like as described above.

The invention may be adapted in various ways (not shown in the diagrams) to situations where multiple monitors are provided at a workstation. It is possible for example to use multiple fan/filter units such as 26 or 52 (the latter with air inlets on each screen) to draw air from the space in front of and below the multiple screens. The embodiment shown in FIG. 15 may be adapted by using a suitably longer version of inlet assembly 216 to span the width of multiple monitors. Gaps between adjoining upright edges of monitors should be suitably blocked to avoid leakage of air from the working space through such gaps.

Note that representations of the various air inlets (28, 122, 222,252, 254) in the drawings are not intended to limit their detailed design features to those shown.

Persons skilled in the art will understand that various design features may be used to prevent ingress of unwanted objects, and help to guide air flow efficiently.

1. An apparatus for treating air from a working space that lies above a work surface and in front of a user of the working space, the apparatus comprising:
an air handling unit;
a fan that draws air through an air inlet into the air handling unit, the air inlet in use being located on an opposite side of the work surface from the user and drawing air from the working space above the work surface and at least partly from below the level of the user's face;
a filter within the air handling unit through which air drawn into the air handling unit passes; and
an air outlet through which air leaving the filter is expelled, the air outlet being positioned outside the working space.

2. The apparatus of claim 1, wherein the working space is further bounded in part by an upright surface in front of the user and the work surface is between the upright surface and the user, and wherein the air inlet is positioned at the upright surface or closer to the upright surface than the user.

3. The apparatus of claim 2, wherein the upright surface comprises a screen surface of a computer monitor assembly and at least a portion of the air inlet is in use positioned below the computer monitor assembly.

4. The apparatus of claim 3, wherein the air handling unit is adapted to lie below the computer monitor assembly and to support the computer monitor assembly.

5. The apparatus of claim 4, wherein the air inlet is comprised in the air handling unit.

6. The apparatus of claim 5, wherein a gap between the computer monitor assembly is closed by a screen extending between the air handling unit and the computer monitor assembly.

7. The apparatus of claim 2, wherein the upright surface comprises the screen surface of a computer monitor assembly and the air inlet wholly or partially surrounds the screen portion of a computer monitor assembly.

8. The apparatus of claim 3, wherein in use the air outlet is positioned behind the computer monitor assembly.

9. The apparatus of claim 2, wherein:
the upright surface comprises a screen surface of a laptop computer and the working surface comprises a keyboard portion of the laptop computer; and
the air inlet is in use positioned on the work surface further from the user than the keyboard portion and below the screen surface.

10. The apparatus of claim 9, wherein the air inlet is in use positioned at a re-entrant corner between the work surface and a portion of the laptop computer comprising the screen of the laptop computer.

11. The apparatus of claim 1, wherein the air inlet is elongate in a direction transverse to the user when the user is facing the work surface.

12. A work station having a working space that lies above a work surface and in front of a user of the work station, the work station comprising:
an air handling unit;
a fan that draws air through an air inlet into the internal space of the air handling unit, the air inlet in use being located on an opposite side of the work surface from the user and drawing air from the working space above the work surface and at least partly from below the level of the user's face;
a filter within the air handling unit through which air drawn into the air handling unit passes; and
an air outlet through which air outlet air leaving the filter is expelled, the air outlet positioned outside the working space.

13. The work station of claim 12, wherein the working space is further bounded in part by an upright surface in front of the user and the work surface is between the upright surface and the user, and wherein the air inlet is positioned at the upright surface or closer to the upright surface than the user.

14. The work station of claim 13, wherein the upright surface comprises a wall or partition.

15. The work station of claim 12, wherein:
the air inlet is one of at least two air inlets, a first of the two air inlets being one positioned to the left of the user and a second of the two air inlets being one positioned to the right of the user, whereby to draw air from both sides of the working space.

16. The work station of claim 12, wherein the air inlet is elongate in a direction transverse to the user when the user is facing the work surface.

17. The A work station according of claim 12, wherein the air handling unit is positioned at a height less than the height of the work surface.

18. The work station of claim 16, wherein the air outlet is directed horizontally and away from a plane that includes the upright surface.

19. A method for treatment of air from a working space that lies above a work surface and in front of a user of the working space, the method comprising:
by a fan, drawing air through an air inlet into an air handling unit, the air inlet being located on an opposite side of the work surface from the user and drawing air from the working space above the work surface and at least partly from below the level of the user's face;
passing air drawn into the air handling unit through a filter within the air handling unit; and
thereafter expelling air leaving the filter from an air outlet outside the working space.

20. The method of claim 19, wherein the working space is further bounded in part by an upright surface in front of the user and the work surface is between the user and the upright surface, and including the step of locating the air inlet closer to the upright surface than a face of the user.
